(19) United States
(12) Patent Application Publication
(10) Pub. No.: US 2014/0120598 A1
(43) Pub. Date: May 1, 2014

(54) Novel Method For Producing Ethanol

(71) Applicants: Akihiko Kondo, Kobe-shi (JP); Tomohisa Hasunuma, Kobe-shi (JP)

(72) Inventors: Akihiko Kondo, Kobe-shi (JP); Tomohisa Hasunuma, Kobe-shi (JP)

(73) Assignee: National University Corporation KOBE University, Hyogo (JP)

(21) Appl. No.: 14/128,633

(22) PCT Filed: Jun. 25, 2012
(86) PCT No.: PCT/JP2012/066115

§ 371 Date: Jan. 17, 2014

(30) Foreign Application Priority Data
Jul. 1, 2011 (JP) 2011-146931

(51) Int. Cl. C12P 7/10

(52) U.S. Cl. CPC C12P 7/10
USPC 435/165; 435/471; 435/254.21; 435/254.23; 435/254.22

(57) ABSTRACT

Provided is a novel method of producing ethanol by using a cellulose-based biomass as a raw material. In particular, provided is a novel method of producing ethanol by which ethanol can be effectively produced in the presence of a substance having an inhibitory action on fermentation of ethanol. Ethanol can be effectively produced by using a microorganism engineered to suppress the expression of at least one kind of phosphatase among the phosphatases intrinsically possessed by the microorganism, even under a condition where a substance that has heretofore been believed to have a fermentation inhibitory action, specifically, a weakly acidic substance and/or a furan compound are/is incorporated.

### TECHNICAL FIELD

The present invention relates to a novel method of producing ethanol by using a cellulose-based biomass as a raw material, and more particularly, to a novel method of producing ethanol by which ethanol can be effectively produced in the presence of a substance having an inhibitory action on fermentation of ethanol.

The present application claims priority of Japanese Patent Application No. 2011-146931, which is incorporated herein by reference.

### BACKGROUND ART

A biomass is a biotic resource present in a large amount such as a tree, grass, seaweed, agricultural waste, or forest industry waste. The amount in which a biomass fuel such as ethanol produced from the biomass can be supplied is limited because the same portion as an edible portion such as the sugar or starch of corn or sugarcane is used as a raw material. In view of the foregoing, the production of bioethanol with waste wood, thinnings, or the like may be extremely advantageous in terms of cost. The development of raw materials that are not edible such as celluloses as main components for the fibers of plants has been advanced. However, it is believed to be technically difficult to produce ethanol from the celluloses as compared to corn or the like because a crystallized cellulose fiber needs to be hydrolyzed into the form of a monosaccharide or disaccharide that can be utilized for a fermentation microorganism such as yeast.

During pretreatments for cellulose-based biomasses, fermentation inhibitors such as weak acids, furfurals, and phenols are necessarily produced in an acidic treatment, a hydrothermal treatment, and the like. In addition, xylitol is a substance useful as a sweetener or the like, and its fermentation production from a polysaccharide-based biomass has been attempted. In this case, however, a problem in that the production of a fermentation inhibitor causes a reduction in yield occurs. Various investigations have been conducted on a method by which a fermentation inhibitor can be efficiently separated from a sugar solution containing the fermentation inhibitor (Patent Literatures 1 and 2).

When a microorganism that ferments a microorganism to produce ethanol (hereinafter sometimes simply referred to as “microorganism for ethanol production”) is a yeast, glucose or fructose is most effective as a carbon source for ethanol. However, a biomass raw material contains various saccharides as carbon sources and also contains a large amount of xylose. A yeast (Saccharomyces cerevisiae) improved as described below has been reported (Non Patent Literatures 1 to 3). For example, the yeast overexpresses a xylulokinase and has a xylose reductase gene or xylitol dehydrogenase gene added thereto so that xylose can also be effectively utilized as a carbon source in ethanol production. In addition, a yeast from which PHO13 as one kind of alkaline phosphatase has been knocked out among such yeast has been reported, and it has been reported that the yeast from which PHO13 has been knocked out is excellent in ability to produce ethanol from xylose (Non Patent Literature 2).

However, a method of producing ethanol from a cellulose-based biomass by microbial fermentation has involved the following problem. A weakly acidic substance, furan compound, or the like to be produced as a by-product in the step of pretreating the cellulose-based biomass cannot be easily removed, and hence the production of bioethanol is not easily performed.

### CITATION LIST

### Patent Literature

[PTL 1] JP 2005-270056 A

[PTL 2] JP 2011-078327 A Non Patent Literature

[NPL 1] APPLIED AND ENVIRONMENTAL MICROBIOLOGY, 67, 4249-4255 (2001)

[NPL 2] Metabolic Engineering, 10, 360-369 (2008)

[NPL 3] Microbial Cell Factories 2011, 10:2

### SUMMARY OF INVENTION

### Technical Problem

An object of the present invention is to provide a novel method of producing ethanol by using a cellulose-based biomass as a raw material. In particular, the object of the present invention is to provide a novel method of producing ethanol by which ethanol can be effectively produced in the presence of a substance having an inhibitory action on the fermentation of ethanol.

### Solution to Problem

The inventors of the present invention have made extensive studies to solve the problems. As a result, the inventors have found that ethanol can be effectively produced by using a microorganism engineered to suppress the expression of at least one kind of phosphatase among the phosphatases intrinsically possessed by the microorganism, even under a condition where a substance that has heretofore been believed to have a fermentation inhibitory action, specifically, such a weakly acidic substance and/or furan compound that ethanol production is inhibited in the case of a conventional microorganism are/is incorporated. Thus, the inventors have completed the present invention.

That is, the present invention includes the following.

1. A method of producing ethanol by using a cellulose-based biomass as a raw material through a microbial fermentation, the method including fermenting the biomass with a microorganism engineered to suppress expression of at least one kind of phosphatase among phosphatases intrinsically possessed by the microorganism under a condition where a weakly acidic substance and/or furan compound having a fermentation inhibitory action are/is incorporated.
2. A method of producing ethanol according the above-mentioned item 1, in which the suppression of the expression of the at least one kind of phosphatase is achieved by deleting part or an entirety of at least one kind of phosphatase gene among phosphatase genes present on a genome of the microorganism.
3. A method of producing ethanol according the above-mentioned item 1 or 2, in which the phosphatase whose expression is suppressed includes at least one kind of phosphatase selected from phosphatases consisting of APM3, PHO2, APL5, APL6, PHO4, PHO13, PHO85, PHO80, PHO9, PHO5, and PHO81.
4. A method of producing ethanol according the above-mentioned item 3, in which the phosphatase whose expression is suppressed includes at least one kind of phosphatase selected from phosphatases consisting of PHO2, PHO13, APL5, and APL6.
5. A method of producing ethanol according to any one of the above-mentioned items 1 to 4, in which the weakly acidic substance includes at least one kind of substance selected from acetic acid and formic acid.
6. A method of producing ethanol according the above-mentioned item 5, in which the fermentation is performed under a condition where 10 mM to 100 mM of acetic acid are incorporated.
7. A method of producing ethanol according the above-mentioned item 5, in which the fermentation is performed under a condition where 5 mM to 50 mM of formic acid are incorporated.
8. A method of producing ethanol according to any one of the above-mentioned items 1 to 7, in which the furan compound includes furfural.
9. A method of producing ethanol according the above-mentioned item 8, in which the fermentation is performed under a condition where 10 mM to 100 mM of furfural are incorporated.
10. A method of producing ethanol according to any one of the above-mentioned items 1 to 9, in which the microorganism includes a yeast belonging to a genus Saccharomyces.
11. A method of producing ethanol according the above-mentioned item 10, in which the yeast belonging to the genus Saccharomyces includes a xylose-assimilating yeast.
12. A microorganism to be utilized in the method of producing ethanol according to any one of the above-mentioned items 1 to 11, in which part or an entirety of at least one kind of phosphatase gene among phosphatase genes present on a genome thereof is deleted.
13. A method of producing a microorganism that produces ethanol by using, as a raw material, a biomass-saccharified liquid containing one or more kinds of fermentation inhibitors selected from acetic acid, formic acid, and furfural, the method including deleting part or an entirety of at least one kind of phosphatase gene among phosphatase genes present on a genome of the microorganism.
14. A method of producing a microorganism according the above-mentioned item 13, in which the fermentation inhibitor in the biomass-saccharified liquid includes one or more kinds selected from 10 mM to 100 mM of acetic acid, 5 mM to 50 mM of formic acid, and 10 mM to 100 mM of furfural.
15. A method of producing a microorganism according the above-mentioned item 13 or 14, in which the microorganism includes a xylose-assimilating yeast belonging to a genus Saccharomyces.

### Advantageous Effects of Invention

In the method of the present invention including producing ethanol by using a cellulose-based biomass as a raw material through a microbial fermentation, ethanol can be effectively produced even through fermentation under a condition where a weakly acidic substance and/or furan compound having a fermentation inhibitory action are/is incorporated by using a microorganism engineered to suppress the expression of at least one kind of phosphatase among the phosphatases intrinsically possessed by the microorganism. Therefore, in the case of the cellulose-based biomass raw material, the removal of a fermentation inhibitor has heretofore been a problem and its operation has been complicated, but according to the method of the present invention, ethanol can be simply produced from the biomass raw material even in the presence of the fermentation inhibitor.

### BRIEF DESCRIPTION OF DRAWINGS

FIG. 1 are graphs confirming the consumption of glucose and xylose, and ethanol-producing ability in a system containing acetic acid as a fermentation inhibitor or a system free of acetic acid (Reference Example 2).

FIG. 2 are graphs confirming, for a yeast (S. cerevisiae) having a xylose-assimilating ability, an alcohol-producing ability when xylose is used as a carbon source by using a strain from which an alkaline phosphatase (PHO13) has been deleted (ΔPHO13 strain) (Reference Example 3).

FIG. 3 are graphs confirming an ability to produce an alcohol (ethanol or xylitol) from a biomass-saccharified liquid with the ΔPHO13 strain (Example 1).

FIG. 4 are graphs confirming the ability of the ΔPHO13 strain to produce ethanol in the presence of acetic acid (Example 2).

FIG. 5 are graphs confirming the ability of the ΔPHO13 strain to produce ethanol in the presence of formic acid (Example 3).

FIG. 6 are graphs confirming the ability of the ΔPHO13 strain to produce ethanol in the presence of furfural (Example 4).

FIG. 7 are graphs confirming ethanol-producing abilities when xylose is used as a carbon source by using various phosphatase gene-deleted strains (Example 5).

FIG. 8 are graphs confirming ethanol-producing abilities in systems using xylose as a carbon source and containing acetic acid by using various phosphatase gene-deleted strains (Example 5).

### DESCRIPTION OF EMBODIMENTS

The present invention relates to a method of producing ethanol by using a cellulose-based biomass as a raw material through a microbial fermentation, the method being characterized by including fermenting the biomass with a microorganism engineered to suppress the expression of at least one kind of phosphatase among the phosphatases intrinsically possessed by the microorganism under a condition where a weakly acidic substance and/or furan compound having a fermentation inhibitory action are/is incorporated.

The term “cellulose-based biomass” as used herein refers to a biomass containing a cellulose of a polysaccharide constructing a plant cell wall, and generally refers to a tree, grass, an agricultural product, the non-edible portion of the agricultural product, and the residue of the agricultural product. In addition, examples thereof include construction waste, thinnings, rice straw, a reed, straw, bagasse (sugarcane residue), napier grass, Erianthus, Miscanthus, and stems and leaves of corn. The cellulose-based biomass is mainly formed of a cellulose, a hemicellulose, and lignin. The cellulose is a polysaccharide formed by dehydration condensation of glucose, which is a typical monosaccharide, and the hemicellulose is a heteropolysaccharide formed by dehydration condensation of, for example, glucose, xylose, and mannose. It is difficult to utilize lignin as a biomass raw material because lignin is a phenolic compound and hard to decompose. Accordingly, a treatment for the removal of lignin may be performed in a pretreatment step.

In the method of producing ethanol of the present invention, the cellulose-based biomass can be pretreated before use. A method known per se or any method to be developed in the future can be applied as a method for the pretreatment. For example, the cellulose-based biomass can be cut and pulverized, and then subjected to a hydrothermal treatment under a high-temperature condition of 130 to 300° C. and under a high-pressure condition of up to 10 MPa to provide a “cellulose-based biomass partially decomposed product” in which the biomass is swollen with moisture and partially decomposed.

The cellulose-based biomass partially decomposed product contains a cellulose or hemicellulose of a plant. The cellulose or the hemicellulose can be saccharified by being decomposed into glucose, xylose, arabinose, cellobiose, mannose, galactose, uronic acid, or o-methyl-uronic acid, or an oligosaccharide in which 2 to 9 of these saccharides are connected or a polysaccharide in which 10 or more thereof are connected through an enzymatic treatment or the like. A treatment method involving decomposing the cellulose or the hemicellulose into various saccharides to saccharify the cellulose or the hemicellulose is not limited to the enzymatic treatment, and a method known per se or any method to be developed in the future can be applied. Thus, a raw material that can be used in the fermentation of a microorganism for ethanol production can be prepared. A raw material that can be used in the method of producing ethanol of the present invention has only to be derived from the cellulose-based biomass, and may be subjected to any pretreatment as long as the raw material can be used in ethanol production. Hereinafter, in the description, a cellulose-based biomass-saccharified liquid as a raw material that can be used in the fermentation of the microorganism for ethanol production is simply referred to as “cellulose-based biomass-saccharified liquid.”

In the description, ethanol can be produced by: adding the microorganism for ethanol production to the cellulose-based biomass-saccharified liquid; and cultivating the microorganism under proper conditions such as a temperature (15 to 50° C.) and a pH (3.0 to 9.0) to ferment the microorganism to transform a saccharide into ethanol. At this time, a microorganism fermentation substrate such as nitrogen or phosphorus may be further added to the cellulose-based biomass-saccharified liquid as required.

In the production of ethanol involving using the cellulose-based biomass as a raw material through the microbial fermentation, examples of a fermentation inhibitor that may reduce the yield of ethanol include various fermentation inhibitors such as: weakly acidic substances such as acetic acid and formic acid produced as by-products in the treatment step for obtaining the cellulose-based biomass partially decomposed product; furan compounds such as furfural and 5-hydroxymethylfurfural; and various phenolic compounds derived from lignin such as guaiacol, vanillin, and syringaldehyde. However, a weakly acidic substance and a furan compound cause problems in terms of the amounts in which the inhibitors are produced as by-products and their inhibitory actions. A fermentation inhibitory action by a weakly acidic substance such as acetic acid or formic acid is remarkable particularly when ethanol is produced by using xylose as a carbon source.

In the description, a “weakly acidic substance having a fermentation inhibitory action” is, for example, acetic acid and/or formic acid, and a “furan compound having a fermentation inhibitory action” is, for example, furfural. The term “condition where a weakly acidic substance and/or furan compound having a fermentation inhibitory action are/is incorporated” as used herein refers to, for example, a condition where acetic acid is incorporated in an amount of 10 mM to 100 mM, preferably 10 mM to 60 mM, more preferably 10 mM to 30 mM. Similarly, the term refers to a condition where formic acid is incorporated in an amount of 5 mM to 50 mM, preferably 5 mM to 30 mM, more preferably 5 mM to 15 mM. Similarly, the term refers to a condition where furfural is incorporated in an amount of 10 mM to 100 mM, preferably 10 mM to 90 mM, more preferably 10 mM to 60 mM.

The phrase “fermented under a condition where a weakly acidic substance and/or furan compound having a fermentation inhibitory action are/is incorporated” as used herein means that the microorganism for ethanol production is added to the cellulose-based biomass-saccharified liquid containing the weakly acidic substance and/or furan compound having a fermentation inhibitory action, and the microorganism is cultivated under conditions such as a temperature (15 to 50° C.) and a pH (3.0 to 9.0) to be fermented. In ordinary cases, “under the condition where the weakly acidic substance and/or furan compound having a fermentation inhibitory action are/is incorporated,” the fermentation of the microorganism is inhibited and hence ethanol cannot be effectively produced. However, according to the method of producing ethanol of the present invention, ethanol can be effectively produced even under the condition where the weakly acidic substance and/or furan compound having a fermentation inhibitory action are/is incorporated.

Examples of the microorganism that can be used in the method of producing ethanol of the present invention include conventionally known various microorganisms for ethanol production belonging to yeasts of the genus Saccharomyces, yeasts of the genus Pichia, yeasts of the genus Candida, and yeasts of the genus Scheffersomyces. Preferred examples thereof include yeasts belonging to the genus Saccharomyces. More preferred examples thereof include xylose-assimilating yeasts belonging to the genus Saccharomyces. Specific examples of the xylose-assimilating yeasts belonging to the genus Saccharomyces include yeasts described in Non Patent Literatures 1 to 3. The use of the xylose-assimilating yeast enables effective utilization of even xylose as a carbon source in ethanol production.

The microorganism that can be used in the description is the microorganism for ethanol production and the expression of at least one kind of phosphatase among the phosphatases intrinsically possessed by the microorganism needs to be suppressed. Examples of the “phosphatases intrinsically possessed by the microorganism” in the description include APM3, PHO2, APL5, APL6, PHO4, PHO13, PHO85, PHO80, PHO9, PHO5, and PHO81. The at least one kind of phosphatase is at least one kind of phosphatase selected from the phosphatases listed above and is suitably at least one kind of phosphatase selected from phosphatases consisting of PHO2, PHO13, APL5, and APL6.

The phrase “the expression of at least one kind of phosphatase is suppressed” as used herein can mean that the microorganism is engineered to suppress the expression of the at least one kind of phosphatase. “Such engineering that the expression of the phosphatase is suppressed” has only to be a method by which the expression of the phosphatase is suppressed, and is not particularly limited. For example, part or the entirety of a gene that encodes the phosphatase (simply referred to as “phosphatase gene”) may be deleted, or a region including a promoter or the like may be modified so that the gene may not be expressed. The use of the microorganism engineered to suppress the expression of the at least one kind of phosphatase enables the production of ethanol under a condition where the weakly acidic substance and/or furan compound that have/has heretofore been said to be the so-called fermentation inhibitors/inhibitor are/is incorporated.

The present invention also encompasses a microorganism that can be used in the method of producing ethanol of the present invention. The microorganism that can be used in the method of producing ethanol of the present invention, i.e., a microorganism capable of producing ethanol in the presence of a weakly acidic substance and/or furan compound having a fermentation inhibitory action refers to a microorganism capable of producing ethanol by: adding the microorganism to a biomass-saccharified liquid containing the weakly acidic substance and/or furan compound having a fermentation inhibitory action; and cultivating the microorganism under proper conditions such as a temperature (15 to 50° C.) and a pH (3.0 to 9.0). The weakly acidic substance having a fermentation inhibitory action is, for example, acetic acid and/or formic acid described above, and the furan compound is, for example, furfural. More specifically, the microorganism refers to a microorganism capable of producing ethanol by: adding the microorganism to a biomass-saccharified liquid containing one or more kinds of fermentation inhibitors selected from 10 mM to 100 mM of acetic acid, 5 mM to 50 mM of formic acid, and 10 mM to 100 mM of furfural; and cultivating the microorganism under proper conditions such as a temperature (15 to 50° C.) and a pH (3.0 to 9.0).

The present invention also encompasses a method of producing a microorganism that can be used in the method of producing ethanol of the present invention. The microorganism that can be used in the method of producing ethanol of the present invention, i.e., a microorganism capable of producing ethanol by adding the microorganism to a biomass-saccharified liquid containing one or more kinds of fermentation inhibitors selected from 10 mM to 100 mM of acetic acid, 5 mM to 50 mM of formic acid, and 10 mM to 100 mM of furfural, and cultivating the microorganism under proper conditions such as a temperature (15 to 50° C.) and a pH (3.0 to 9.0) can be produced by deleting part or the entirety of at least one kind of phosphatase gene among the phosphatase genes present on the genome of the microorganism. A method for such engineering that the expression of the phosphatase is suppressed can be specifically achieved by a method in conformity with a method described in, for example, Non Patent Literature 3.

### EXAMPLES

In order that the understanding of the present invention may be deepened, how the present invention was completed is described in Reference Examples and the contents of the present invention are specifically described by way of Examples. However, it is evident that the present invention is not limited to these examples.

### Reference Example 1

### Fermentation Inhibitors in Biomass-Saccharified Liquid

In this reference example, fermentation inhibitors present in a biomass-saccharified liquid using a rice straw as a raw material and subjected to a hydrothermal treatment (conditions: 130 to 300° C., 1 to 10 MPa) were confirmed. The rice straw was subjected to a hydrothermal treatment and then subjected to solid-liquid separation, followed by the recovery of a liquid fraction. After that, the pH of the liquid fraction was adjusted to 5 with NaOH and then a 1% (w/v) of a hemicellulase (G-Amano; manufactured by Amano Enzyme Inc.) was added to the liquid fraction, followed by a treatment at 37° C. for 72 hours. After that, the treated product was centrifuged at 15,000 g and 4° C. for 60 minutes, and then the supernatant was recovered. The supernatant was defined as a biomass-saccharified liquid.

The fermentation inhibitors, such as acetic acid, formic acid, furfural, 5-hydroxymethyl-2-furfural (5-HMF), vanillin, o-vanillin, eugenol, isoeugenol, and syringaldehyde, in the saccharified liquid were measured by gas chromatography-mass spectrometry (GC-MS) (QP2010Plus, Shimadzu Corporation). The acids were measured with a capillary column (DB-FFAP column, 60 m×0.25 mm, film thickness: 0.5 μm; Agilent Technologies). The furan compounds and phenols were measured with a capillary column (CP-Sil 8-CB low Bleed/MS column, 30 m×0.25 mm, film thickness: 0.25 μm; Varian, Inc.).

TABLE 1
Fermentation inhibitor in biomass-decomposed liquid

| | [mM] |
|---|---|
| Acetic acid (Acetate) | 27.11 |
| Formic acid (Formate) | 20.06 |
| Furfural | 7.77 |
| 5-HMF | 0.46 |
| Vanillin | 0.56 |
| Syringaldehyde | 0.37 |

### Reference Example 2

### Consumption of Various Carbon Sources and Production of Alcohol in the Presence of Acetic Acid

In this reference example, the consumption of various carbon sources and alcohol-producing ability in a yeast (S. cerevisiae MN8140X: Non Patent Literature 3) to which a xylose-assimilating ability had been imparted were confirmed in each of the case where acetic acid was added to a solution using glucose and xylose as carbon sources, and the case where acetic acid was not added to the solution as model systems of a biomass-saccharified liquid. A medium formed of 10 g/L of a yeast extract, 20 g/L of polypeptone, 80 g/L of glucose, and 60 g/L of xylose was used as the solution using glucose and xylose as carbon sources. The cells were added to the medium so as to have an initial concentration of 50 g/L and then a fermentation treatment was performed at a fermentation temperature of 30° C.

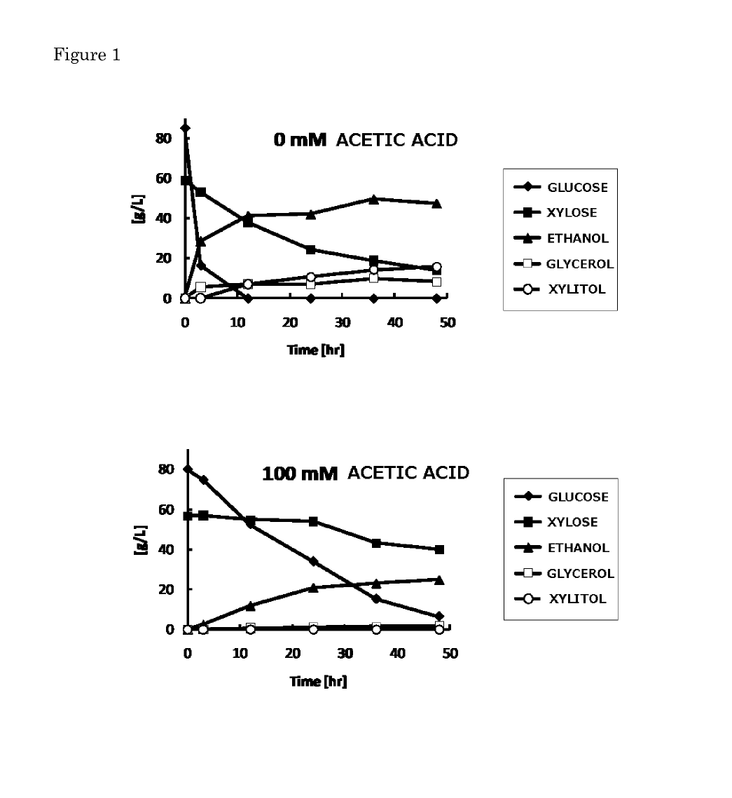

FIG. 1 show results when the fermentation treatment was performed for 48 hours under the above-mentioned conditions. It was confirmed that when the solution contained 100 mM of acetic acid, the consumption of glucose as a carbon source was suppressed and the amount of production of ethanol was also suppressed. It was confirmed from the foregoing that acetic acid showed a fermentation inhibitory action in ethanol production by the yeast.

### Reference Example 3

### Re: Xylose-Assimilating Ability of Alkaline Phosphatase-Deleted Strain

In this reference example, an assimilating ability when xylose was used as a carbon source was confirmed for an S. cerevisiae BY4741X strain (hereinafter referred to as “BY4741X strain”), which was obtained by imparting a xylose-assimilating ability to an S. cerevisiae BY4741 strain according to the same method as the method disclosed in Non Patent Literature 3, by using a strain from which an alkaline phosphatase (PHO13) had been deleted (hereinafter sometimes referred to as “ΔPHO13 strain”). A yeast (BY4741X strain) to which a xylose-assimilating ability had been merely imparted and from which PHO13 had not been deleted was used as a control.

A solution obtained by incorporating 80 g/L of xylose into a YP medium (containing 1% of a yeast extract, 2% of peptone, and 0.5% of dipotassium disulfite) was used as a material using xylose as a carbon source. Each of the cells was added to the solution so as to have an initial concentration of 50 g/L and then a fermentation treatment was performed at a fermentation temperature of 30° C.

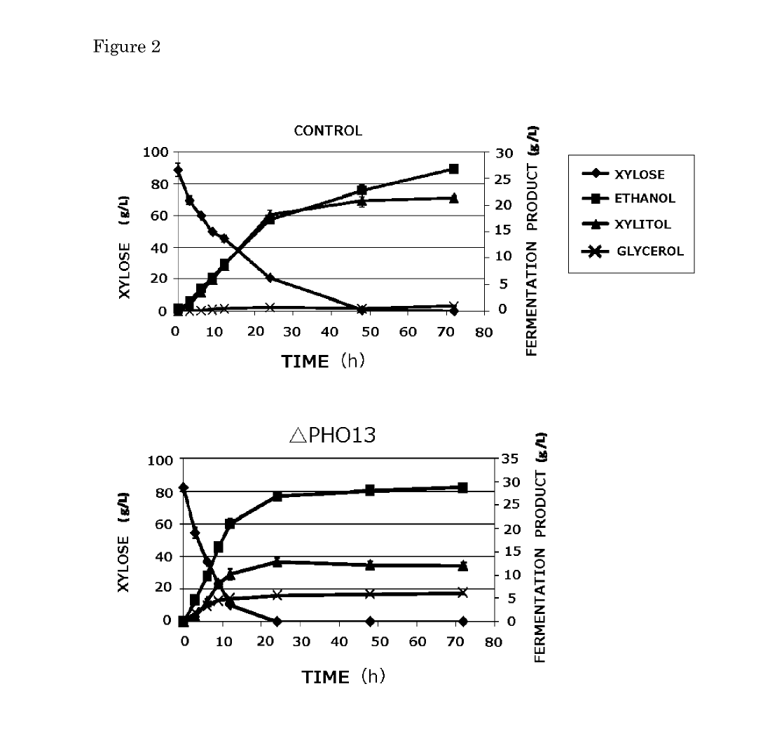

FIG. 2 show results when the fermentation treatment was performed for 72 hours under the above-mentioned conditions. It was confirmed that the PHO13 strain had a faster consumption rate of xylose than that of the control. In addition, while the amount of production of ethanol reached amaximumamount of 30 g/L after 24 hours of cultivation in the case of the ΔPHO13 strain, the amount of production was 27 g/L even after 72 hours of cultivation in the case of the control.

### Example 1

### Consumption of Various Carbon Sources and Production of Alcohol in Biomass-Saccharified Liquid

In view of the fact that the PHO13 strain was confirmed to have an excellent ethanol-producing ability, whether ethanol could be produced by consuming, for example, glucose and fructose, or xylose even in the presence of a fermentation inhibitory active material was confirmed for the ΔPHO13 strain. A biomass-saccharified liquid containing various fermentation inhibitors shown in Reference Example 1 (Table 1) was used as a raw material and a fermentation treatment was performed in accordance with the fermentation conditions described in Reference Example 2. The BY4741X strain was used as a control as in Reference Example 3.

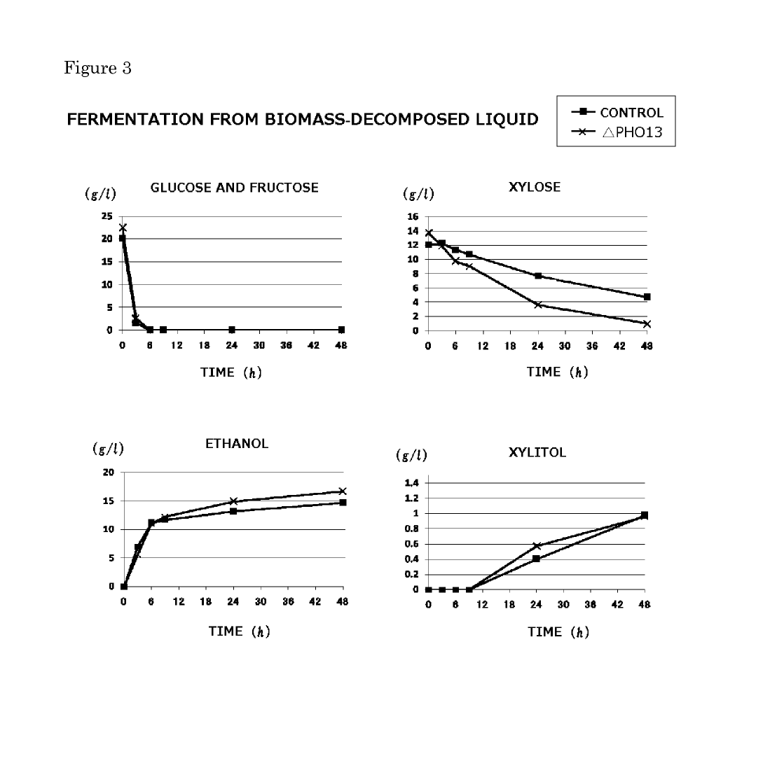

FIG. 3 show results when the fermentation treatment was performed for 48 hours under the above-mentioned conditions. The consumption rates of glucose and fructose were fast because the yeast fungi had assimilating actions on these saccharides. On the other hand, the ΔPHO13 strain had a faster consumption rate of xylose as that of the control. The ΔPHO13 strain was more excellent in abilities to produce ethanol and xylitol.

### Example 2

### Re: Xylose-Assimilating Ability in the Presence of Acetic Acid

In this example, the extent to which the ΔPHO13 strain could produce ethanol by consuming xylose even in the presence of acetic acid having fermentation inhibitory activity was compared to a control (BY4741X strain). An investigation was conducted by using a material using xylose as a carbon source in the same manner as in Reference Example 3 except that a fermentation condition was changed as follows: acetic acid was added at a concentration of each of 0, 30, and 60 mM.

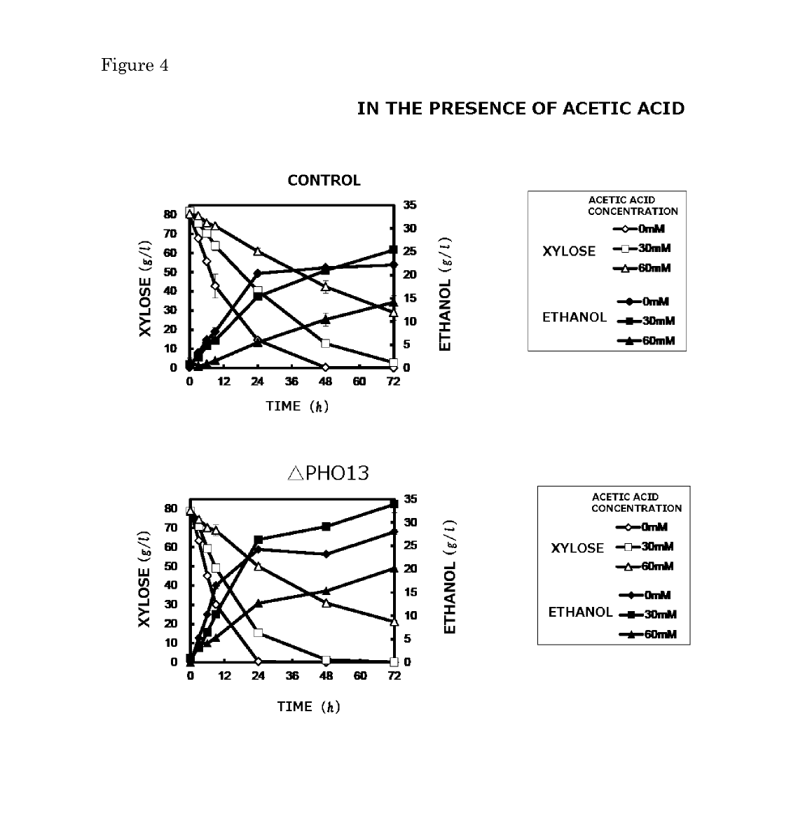

FIG. 4 show results when the fermentation treatment was performed for 72 hours under the above-mentioned conditions. In each of the control and the ΔPHO13 strain, the consumption rate of xylose reduced depending on an acetic acid concentration. However, the ΔPHO13 strain had a faster consumption rate of xylose at each acetic acid concentration. In addition, the ΔPHO13 strain had a larger amount of production of ethanol at each acetic acid concentration. In particular, in the presence of 60 mM of acetic acid, the amount of production was 13 g/L after a lapse of 24 hours (2.3 times as large as that of the control) and was 20 g/L after a lapse of 72 hours (1.4 times as large as that of the control). The ΔPHO13 strain was found to be resistant to acetic acid having a fermentation inhibitory action for ethanol production involving using xylose as a carbon source.

### Example 3

### Re: Xylose-Assimilating Ability in the Presence of Formic Acid

In this example, a fermentation treatment was performed to confirm an ethanol-producing ability in the same manner as in Example 2 except that formic acid was added at a concentration of each of 0, 15, and 30 mM.

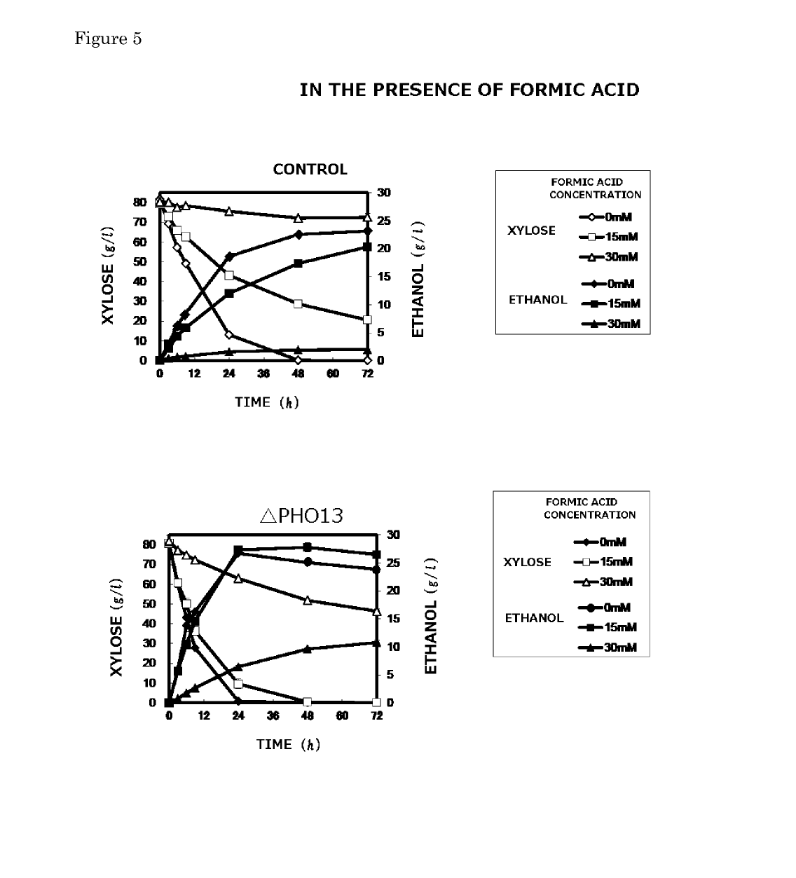

FIG. 5 show results when the fermentation treatment was performed for 72 hours under the above-mentioned conditions. In the case of formic acid as well, a tendency similar to that in the case of acetic acid was observed. In particular, in the presence of 30 mM of formic acid, the ΔPHO13 strain produced ethanol in amounts of 6 g/L after a lapse of 24 hours (4.1 times as large as that of the control) and 11 g/L after a lapse of 72 hours (5.5 times as large as that of the control), respectively. The ΔPHO13 strain was found to be resistant to formic acid having a fermentation inhibitory action for ethanol production involving using xylose as a carbon source.

### Example 4

### Re: Xylose-Assimilating Ability in the Presence of Furfural

In this example, a fermentation treatment was performed to confirm an ethanol-producing ability in the same manner as in Example 2 except that furfural was added at a concentration of each of 0, 60, and 90 mM.

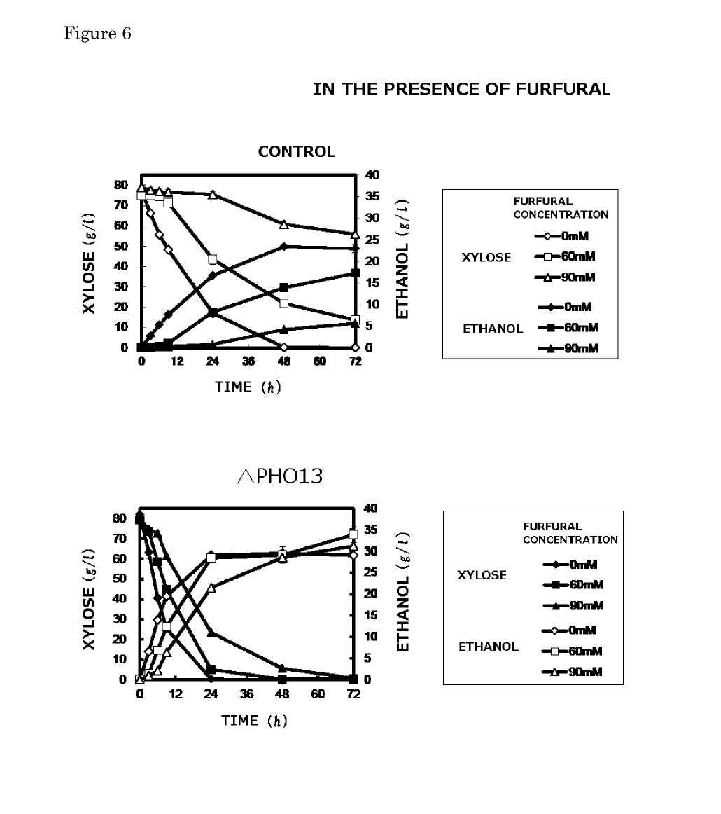

FIG. 6 show results when the fermentation treatment was performed for 72 hours under the above-mentioned conditions. In the control, the consumption rate of xylose reduced in the case where furfural was incorporated. In contrast, in the ΔPHO13 strain, the consumption rate of xylose in the case where 60 mM of furfural were incorporated was substantially the same as that in the case where furfural was not incorporated. Meanwhile, with regard to the ethanol-producing ability, effective ethanol production was similarly observed in the case where 60 mM of furfural were incorporated and the case where furfural was not incorporated. In addition, in the presence of 90 mM of furfural, the ΔPHO13 strain produced ethanol in amounts of 21 g/L after a lapse of 24 hours (27.5 times as large as that of the control) and 31 g/L after a lapse of 72 hours (5.5 times as large as that of the control), respectively. The ΔPHO13 strain was found to be resistant to furfural having a fermentation inhibitory action for ethanol production involving using xylose as a carbon source.

### Example 5

### Re: Xylose-Assimilating Abilities in Various Phosphatase-Deleted Strains

In this example, ethanol-producing abilities in the case where 30 mM of acetic acid were added and the case where acetic acid was not added were compared for various phosphatase-deleted strains. A fermentation treatment was performed to confirm an ethanol-producing ability in the same manner as in Example 2 except that yeast fungi from which the following various phosphatase genes had been deleted were used.

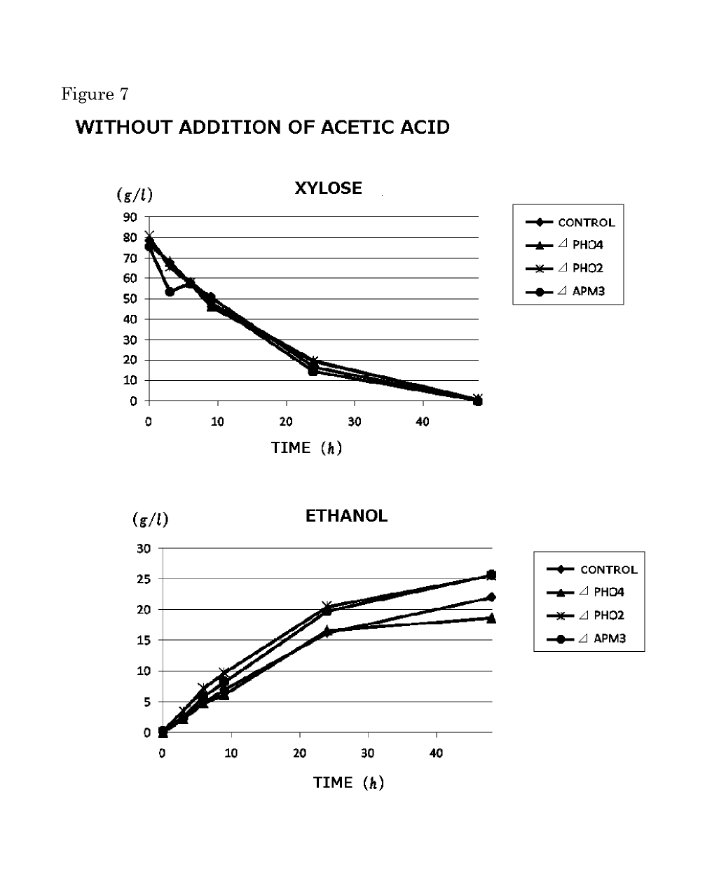

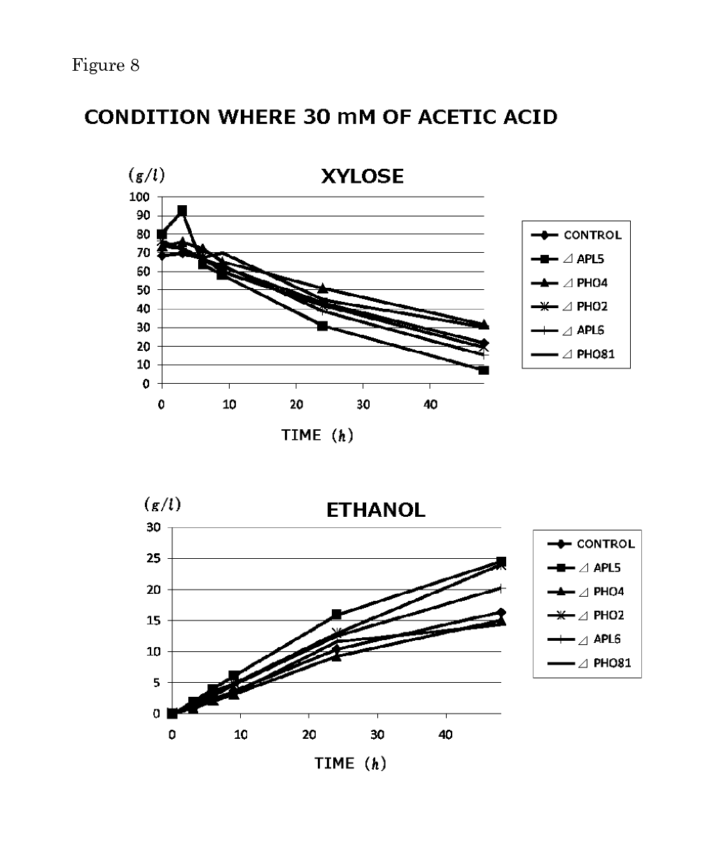

Ethanol-producing abilities were confirmed for yeast strains obtained by deleting genes of various alkali phosphatase (PHO4, PHO2, and APM3) from the BY4741X strain described in Reference Example 3 (a ΔPHO4 strain, a ΔPHO2 strain, and a ΔAPM3 strain, respectively), and the BY4741X strain as a control. As a result, the consumption rates of xylose of the strains were substantially the same as that of the control irrespective of which alkali phosphatase gene had been deleted. However, with regard to the amount of production of ethanol, the ΔPHO2 strain and the ΔAPM3 strain showed more efficient producing abilities than that of the control (FIGS. 7 and 8).

### INDUSTRIAL APPLICABILITY

As described in detail above, in the method of the present invention including producing ethanol by using a cellulose-based biomass as a raw material through the microbial fermentation, ethanol can be effectively produced even through fermentation under a condition where a weakly acidic substance and/or furan compound having fermentation inhibitory action are/is incorporated by using a microorganism engineered to suppress the expression of at least one kind of phosphatase among the phosphatases intrinsically possessed by the microorganism. Therefore, in the case of the cellulose-based biomass raw material, the removal of a fermentation inhibitor has heretofore been a problem and its operation has been complicated, but according to the method of the present invention, ethanol can be simply produced from the biomass raw material even in the presence of the fermentation inhibitor. Accordingly, the method of the present invention is extremely significant.

1. A method of producing ethanol by using a cellulose-based biomass as a raw material through a microbial fermentation, the method comprising fermenting the biomass with a microorganism engineered to suppress expression of at least one kind of phosphatase among phosphatases intrinsically possessed by the microorganism under a condition where a weakly acidic substance and/or furan compound having a fermentation inhibitory action are/is incorporated.

2. A method of producing ethanol according to claim 1, wherein the suppression of the expression of the at least one kind of phosphatase is achieved by deleting part or an entirety of at least one kind of phosphatase gene among phosphatase genes present on a genome of the microorganism.

3. A method of producing ethanol according to claim 1, wherein the phosphatase whose expression is suppressed comprises at least one kind of phosphatase selected from phosphatases consisting of APM3, PHO2, APL5, APL6, PHO4, PHO13, PHO85, PHO80, PHO9, PHO5, and PHO81.

4. A method of producing ethanol according to claim 3, wherein the phosphatase whose expression is suppressed comprises at least one kind of phosphatase selected from phosphatases consisting of PHO2, PHO13, APL5, and APL6.

5. A method of producing ethanol according to claim 1, wherein the weakly acidic substance comprises at least one kind of substance selected from acetic acid and formic acid.

6. A method of producing ethanol according to claim 5, wherein the fermentation is performed under a condition where 10 mM to 100 mM of acetic acid are incorporated.

7. A method of producing ethanol according to claim 5, wherein the fermentation is performed under a condition where 5 mM to 50 mM of formic acid are incorporated.

8. A method of producing ethanol according to claim 1, wherein the furan compound comprises furfural.

9. A method of producing ethanol according to claim 8, wherein the fermentation is performed under a condition where 10 mM to 100 mM of furfural are incorporated.

10. A method of producing ethanol according to claim 1, wherein the microorganism comprises a yeast belonging to a genus Saccharomyces.

11. A method of producing ethanol according to claim 10, wherein the yeast belonging to the genus Saccharomyces comprises a xylose-assimilating yeast.

12. A microorganism to be utilized in the method of producing ethanol according to claim 1, wherein part or an entirety of at least one kind of phosphatase gene among phosphatase genes present on a genome thereof is deleted.

13. A method of producing a microorganism that produces ethanol by using, as a raw material, a biomass-saccharified liquid containing one or more kinds of fermentation inhibitors selected from acetic acid, formic acid, and furfural, the method comprising deleting part or an entirety of at least one kind of phosphatase gene among phosphatase genes present on a genome of the microorganism.

14. A method of producing a microorganism according to claim 13, wherein the fermentation inhibitor in the biomass-saccharified liquid comprises one or more kinds selected from 10 mM to 100 mM of acetic acid, 5 mM to 50 mM of formic acid, and 10 mM to 100 mM of furfural.

15. A method of producing a microorganism according to claim 13, wherein the microorganism comprises a xylose-assimilating yeast belonging to a genus Saccharomyces.
